SEQUENCE LISTING (2 sequences)

SEQ ID NO: 1 - protein, 1 aa, Artificial Sequence
  upregulator of expression of a gene encoding an antioxidative enzyme
X

SEQ ID NO: 2 - protein, 6 aa, Artificial Sequence
  amino terminal capping group
XXXXXX

(19) United States
(12) Patent Application Publication
(10) Pub. No.: US 2005/0130881 A1
(43) Pub. Date: Jun. 16, 2005

(54) Single amino acid based compounds for counteracting effects of reactive oxygen species and free radicals

(76) Inventor: Victor E. Shashoua, Brookline, MA (US)

Correspondence Address: Brown Raysman Millstein;Felder & Steiner, 900 Third Avenue, New York, NY, 10022, US

(21) Appl. No.: 10/478,779

(22) PCT Filed: May 24, 2002
(86) PCT No.: PCT/US02/16768

(30) Foreign Application Priority Data
May 25, 2001 (US) 60293607

(51) Int. Cl. A61K 38/16; A61K 31/385; A61K 38/05; A61K 38/04

(52) U.S. Cl. 514/8; 514/19; 514/440; 514/563; 514/566; 514/17; 514/18

(57) ABSTRACT

Single amino acid compounds and methods for upregulating expression of a gene encoding an antioxidative enzyme, such as superoxide dismutase or catalase, to counteract harmful oxidative effects of reactive oxygen species and other free radicals are described. The single amino acid compounds may be used in compositions and methods to treat or prevent diseases and conditions characterized by undesirable elevation of reactive oxygen species and other free radicals.

### CROSS REFERENCE TO RELATED APPLICATIONS

This application claims priority to U.S. Provisional Application 60/293,607, filed May 25, 2001.

### FIELD OF THE INVENTION

The present invention is in the field of antioxidative compounds. In particular, the invention provides single amino acid based compounds useful in compositions and methods for therapeutic and prophylactic treatments of diseases and conditions, which are characterized by undesirable levels of reactive oxygen species and free radicals.

### BACKGROUND TO THE INVENTION

Biological organisms generate harmful reactive oxygen species (ROS) and various free radicals in the course of normal metabolic activities of tissues such as brain; heart, lung, and muscle tissue (Halliwell, B. and Gutteridge, J. M. C., eds. Free Radicals in Biology and Medicines (Oxford: Clarendon Press, 1989)). The most reactive and, therefore, toxic ROS and free radicals include the superoxide anion (O₂.⁻), singlet oxygen, hydrogen peroxide (H₂O₂), lipid peroxides, peroxinitrite, and hydroxyl radicals. Even a relatively small elevation in ROS or free radical levels in a cell can be damaging. Likewise, a release or increase of ROS or free radicals in extracellular fluid can jeopardize the surrounding tissue and result in tissue destruction and necrosis. Indeed, hydrogen peroxide, which is somewhat less reactive than the superoxide anion, is a well known, broad spectrum, antiseptic compound. In eukaryotes, a major source of superoxide anion is the electron transport system during respiration in the mitochondria. The majority of the superoxide anion is generated at the two main sites of accumulation of reducing equivalents, i.e., the ubiquinone-mediated and the NADH dehydrogenase-mediated steps in the electron transport mechanism. Hydrogen peroxide is generated metabolically in the endoplasmic reticulum, in metal-catalyzed oxidations in peroxisomes, in oxidative phosphorylation in mitochondria, and in the cytosolic oxidation of xanthine (see, e.g., Somani et al., “Response of Antioxidant System to Physical and Chemical Stress,” In Oxidants. Antioxidants, and Free Radicals, chapter 6, pp. 125-141, Baskin, S. I. and H. Salem, eds. (Taylor & Francis, Washington, D.C., 1997)).

In normal and healthy individuals, several naturally occurring antioxidant defense systems detoxify the various ROS or free radicals and, thereby, preserve normal cell and tissue integrity and function. These systems of detoxification involve the stepwise conversion of ROS or free radicals to less toxic species by the concerted activities of certain antioxidative enzymes. These antioxidative enzymes are members of a larger class of molecules known as “oxygen radical scavengers” or “lazaroids” that have an ability to scavenge and detoxify ROS and free radicals. Vitamins A, C, E, and related antioxidant compounds, such as β-carotene, retinoids, and lipoic acid, are also lazaroids. In healthy individuals, sufficient levels of antioxidative enzymes and other lazaroids are present both intracellularly and extracellularly to efficiently scavenge sufficient amounts of ROS and free radicals to avoid significant oxidative damage to cells and tissues.

Superoxide dismutase (SOD), catalase (CAT) and glutathione peroxidase (GSH-Px) are among the most important and studied of the antioxidative enzymes. These enzymes function in concert to detoxify ROS and free radicals. SOD is present in virtually all oxygen-respiring organisms where its major function is the dismutation (breakdown) of superoxide anion to hydrogen peroxide. Hydrogen peroxide, itself, is a highly reactive and oxidative molecule, which must be further reduced to avoid damage to cells and tissues. In the presence of the appropriate electron acceptors (hydrogen donors), CAT catalyzes the further reduction of hydrogen peroxide to water. In the presence of reduced glutathione (GSH), GSH-Px also mediates reduction of hydrogen peroxide to water by a separate pathway.

Each of the antioxidative enzymes described above can be further subdivided into classes. There are three distinct classes of SOD based on metal ion content: copper-zinc (Cu—Zn), manganese (Mn), and iron (Fe). In mammals, only the Cu—Zn and Mn SOD classes are present. Mammalian tissues contain a cytosolic Cu—Zn SOD, a mitochondrial Mn SOD, and a Cu—Zn SOD referred to as EC-SOD, which is secreted into the extracellular fluid. SOD is able to catalyze the dismutation of the highly toxic superoxide anion at a rate that is 10 million times faster than the spontaneous rate (see, Somani et al., p. 126). Although present in virtually all mammalian cells, the highest levels of SOD activity are found in several major organs of high metabolic activity, i.e., liver, kidney, heart, and lung. Expression of the gene encoding SOD has been correlated with tissue oxygenation; high oxygen tension elevates SOD biosynthesis in rats (Toyokuni, S., Pathol. Int., 49: 91-102 (1999)).

CAT is a soluble enzyme present in nearly all mammalian cells, although CAT levels can vary widely between tissues and intracellular locations. CAT is present predominately in the peroxisomes (microbodies) in liver and kidney cells and also in the microperoxisomes of other tissues.

There are two distinct classes of GSH-Px: selenium-dependent and selenium independent. Furthermore, GSH-Px species can be found in as soluble protein in the cytosol, as a membrane-associated protein, and as a circulating plasma protein.

A recognition of the role of ROS and free radicals in a variety of important diseases and drug side effects has grown appreciably over recent years. Many studies have demonstrated that a large number of disease states and harmful side effects of therapeutic drugs are linked with a failure of the antioxidant defense system of an individual to keep up with the rate of generation of ROS and various free radicals (see, e.g., Chan et al., Adv. Neurol., 71:271-279 (1996); DiGuiseppi, J. and Fridovich, I., Crit. Rev. Toxicol., 12:315-342 (1984)). For example, abnormally high ROS levels have been found under conditions of anoxia elicited by ischemia during a stroke or anoxia generated in heart muscle during myocardial infarction (see, e.g., Walton, M. et al., Brain Res. Rev., 29:137-168 (1999); Pulsinelli, W. A. et al., Ann. Neurol., 11: 499-502 (1982); Lucchesi, B. R., Am. J. Cardiol., 65:14I-23I (1990)). An elevation of ROS and free radicals has also been linked with reperfusion damage after renal transplants. Moreover, an increasing number of studies have shown a correlation between oxidative tissue damage and age-associated brain dysfunction, as evidenced by age-related loss of various cognitive and motor functions, and/or a progressive increase in oxidatively modified DNA and proteins during aging (see, e.g., Coyle et al., Science, 262: 689-695 (1993); Halliwell, J. Neurochem., 59: 1609-1623 (1992); Sohal et al., Free Radical Biol. Med., 10: 495-500 (1990); Sohal et al., Mech. Ageing Dev., 76: 215-224 (1994); Ames, Free Radical Res. Commun., 7: 121-128 (1989); and Stadtman, Science, 257:1220-1224 (1992)). Accordingly, an elevation of ROS and free radicals has been linked with the progression and complications developed in many diseases, drug treatments, traumas, and degenerative conditions, including age-related oxidative stress induced damage, age-related loss of motor function, age-related loss of cognitive function, Parkinson's disease, Alzheimer's disease, Huntington's disease, Tardive dyskinesia, degenerative eye diseases, septic shock, head and spinal cord injuries, ulcerative colitis, human leukemia and other cancers, and diabetes (see, e.g., Ratafia, Pharmaceutical Executive, pp. 74-80 (April 1991)).

One approach to reducing elevated levels of damaging ROS and free radicals has involved an attempt to increase the uptake of lazaroids to scavenge or reduce ROS and free radicals. As a result, the commercial market for antioxidative enzymes and other lazaroids is estimated to exceed $1 billion worldwide. Not surprisingly, research and development of various lazaroids as therapeutic agents has become a highly competitive field. Interest in developing SOD itself as a therapeutic agent has been especially strong. This is due, in part, to SOD's status as a recognized anti-inflammatory agent and the belief that SOD might provide a means for penetrating the nonsteroidal, anti-inflammatory drug (NSAID) market as well (Id., at p. 74).

Despite many years of focused research effort, the use of SOD and other lazaroids has not provided a successful prophylactic or therapeutic tool for addressing the diseases, disorders and other conditions caused by or characterized by the generation of ROS and free radicals. Clearly, there remains a need for additional therapeutics and methods of treating diseases and conditions characterized by the destructive effect of elevated levels of ROS and free radicals.

### SUMMARY OF THE INVENTION

The invention described herein solves the problem of how to counteract the destructive oxidative effect of elevated levels of ROS and free radicals by providing compositions comprising single amino acid based compounds that stimulate (i.e., upregulate) expression of genes encoding antioxidative enzymes, such as superoxide dismutase (SOD) and/or catalase (CAT), to reduce, eliminate, or prevent an undesirable elevation in the levels of ROS and free radicals in cells and tissues, and to restore age-related reduction of constitutive antioxidative enzymes. Furthermore, the peptide compounds of this invention may have antioxidative activity independent of their ability to stimulate expression of genes encoding antioxidative enzymes. The formulas and sequences of the peptide compounds described herein use the standard three-letter or one-letter abbreviations for amino acids known in the art.

According to the invention, the amino acid L-aspartic acid, and derivatives thereof, upregulate expression of antioxidative enzymes superoxide dismutase (SOD) and/or catalase (CAT), which counteract the effects of reactive oxygen species and free radicals.

The invention provides compositions comprising a single amino acid based compound having the formula:
R₁-Xaa-R₂ (SEQ ID NO:1),
wherein:
R₁ is absent or is an amino terminal capping group;
Xaa is any amino acid, or derivative thereof, that upregulates expression of a gene encoding an antioxidative enzyme;
R₂ is absent or is a carboxy terminal capping group; and wherein the single amino acid based compound upregulates expression of a gene encoding an antioxidative enzyme.

Preferably, Xaa in the above formula is an amino acid selected from the group consisting of L-aspartic acid, D-aspartic acid, L-asparagine, D-asparagine, L-glutamic acid, D-glutamic acid, L-glutamine, D-glutamine, and derivatives thereof.

In a preferred embodiment, the invention provides compositions comprising single amino acid based compound of the above formula (SEQ ID NO:1) wherein Xaa is L-aspartic acid, L-asparagine, or derivatives thereof, and wherein the single amino acid compound upregulates expression of a gene encoding an antioxidative enzyme. Most preferably, Xaa is L-aspartic acid.

Preferably, the gene(s) for an antioxidative enzyme upregulated by an amino acid compound of the invention encodes superoxide dismutase (SOD) and/or catalase (CAT).

When present, an amino terminal capping group (R₁) useful in the compounds of the invention may be, without limitation, a lipoic acid moiety (Lip), a glucose-3-O-glycolic acid (Gga) moiety, 1 to 6 (preferably 1 or 2) lysine residues (SEQ ID NO:2), 1 to 6 (preferably 1 or 2) arginine residues (SEQ ID NO:2), a lysine and arginine containing peptide of 2-6 amino acid residues (SEQ ID NO:2), an acyl group having the formula R₃—CO—, wherein CO represents a carbonyl group and R₃ is a saturated or an unsaturated (mono- or polyunsaturated) hydrocarbon chain having from 1 to 25 carbons, and combinations thereof. More preferably, the amino terminal capping group is Lip or the R₃—CO— acyl group wherein R₃ is a saturated or unsaturated hydrocarbon chain having 1 to 22 carbons. In another preferred embodiment, the amino terminal capping group is the acyl group that is an acetyl group (Ac), palmitic acid (Palm), or docosahexaenoic acid (DHA).

When present, a carboxy terminal capping group (R₂) useful in the compounds of the invention includes, without limitation, a primary or secondary amine.

The amino acid compounds useful in the compositions and methods of the invention may also be prepared and used as one or more various salt forms, including acetate salts and trifluoroacetic acid salts, depending on the needs for a particular composition or method.

The invention also provides methods of counteracting the effects of ROS and free radicals in cells and tissues comprising contacting the cells or tissues with an amino acid compound described herein. In a preferred embodiment of the invention, the amino acid compounds of the invention stimulate (upregulate) expression of a gene(s) encoding an antioxidative enzyme(s), such as superoxide dismutase (SOD) and/or catalase (CAT) enzymes, which enzymes are capable of detoxifying ROS and free radicals in cells and tissues of animals, including humans and other mammals. Preferably, gene expression for both SOD and CAT proteins are upregulated by contacting cells or tissues with a compound of this invention. Treating cells or tissues with a composition comprising a single amino acid based compound described herein may elevate the expression of a gene(s) encoding SOD and/or CAT to sufficiently high levels to provide significantly increased detoxification of ROS and free radicals compared to untreated cells or tissues.

Individuals having a variety of diseases or conditions have been found to possess undesirable levels of ROS and/or free radicals. In a preferred embodiment of the invention, a composition comprising an amino acid compound described herein may be used therapeutically to counteract the effects of ROS and free radicals present in the body and/or prophylactically to decrease or prevent an undesirable elevation in the levels of ROS and free radicals associated with particular diseases, conditions, drug treatments, or disorders. Specifically, this invention provides methods in which a composition comprising a single amino acid based compound described herein is administered to an animal (i.e., an individual), such as a human or other mammal, to treat or prevent a disease or condition that is characterized by the generation of toxic levels of ROS or free radicals, including but not limited to tissue and/or cognitive degeneration during aging (senescence), senility, Tardive dyskinesia, cerebral ischemia (stroke), myocardial infarct (heart attack), head trauma, brain and/or spinal cord trauma, reperfusion damage, oxygen toxicity in premature infants, Huntington's disease, Parkinson's disease, amyotrophic lateral sclerosis, Alzheimer's disease, diabetes, ulcerative colitis, human leukemia and other cancers characterized by elevation of ROS or free radicals, age-related elevation of ROS or free radicals, Down syndrome, macular degeneration, cataracts, schizophrenia, epilepsy, septic shock, polytraumatous shock, burn injuries and radiation-induced elevation of ROS and free radicals (including UV-induced skin damage).

In a particularly preferred embodiment, this invention provides methods in which a composition comprising a single amino acid based compound described herein is administered to an individual to lessen or eliminate side effects caused by drug regimens that generate ROS and free radicals. A number of drugs have been found to cause undesirable elevation of levels of ROS or free radicals as a toxic side effect. Such drugs include doxorubicin, daunorubicin, BCNU (carmustine) and related compounds such as methyl-BCNU and CCNU, and neuroleptics, such as clozapine. As an adjuvant to such therapies, the amino acid compounds of this invention can be used to decrease the severity of or eliminate these damaging side effects. Accordingly, the amino acid compounds of this invention may be administered to an individual to treat or prevent drug-induced elevation of ROS or free radicals, such as occurs during treatment with neuroleptic drugs as in Tardive dyskinesia.

In yet another embodiment, the amino acid compounds described herein are used as an alternative or adjuvant to nonsteroidal, anti-inflammatory drugs (NSAIDs) to treat pain from wounds, arthritis, and other inflammatory conditions in which ROS and free radicals play a role.

The invention also provides pharmaceutical compositions comprising an amino acid compound of the invention and a pharmaceutically acceptable carrier or buffer for administration to an individual to eliminate, reduce, or prevent the generation of toxic levels of ROS or free radicals in cells or tissues.

Another aspect of the invention provides dietary supplement compositions (also referred to as “nutraceuticals”) comprising a natural source, purified composition obtained or produced from an organism (animal, plant, or microorganism), which contains or is enriched for an endogenous amino acid compound described herein, which upregulates expression of one or more genes encoding an antioxidative enzyme, such as SOD and/or CAT in cells or tissues. Preferably, dietary supplements of the invention additionally comprise an exogenously provided amino acid compound described herein.

### DETAILED DESCRIPTION

This invention is based on the discovery that a single amino acid, such as L-aspartic acid, is capable of upregulating expression of one or both genes encoding a complementary pair of enzymes, i.e., superoxide dismutase (SOD) and catalase (CAT), which are major components of the antioxidative defense mechanism or system in cells and tissues to detoxify reactive oxygen species (ROS) and free radicals. ROS and free radicals are generated during electron transport and normal respiration and other metabolic processes, including during the metabolism of various drugs, and must be rapidly detoxified to prevent permanent and continuing damage to cells and tissues. In addition, a number of diseases or conditions, including the aging process (senescence), have also been characterized by an elevation of ROS and/or free radicals to toxic levels that in fact damage cells and tissues. Accordingly, the single amino acid based compounds described herein are valuable therapeutic and prophylactic compounds for counteracting the generation of harmful levels of ROS and free radicals in an individual.

In order that the invention may be better understood, the following terms are defined.

Abbreviations: Amino acid residues described herein may be abbreviated by the conventional three-letter or one letter abbreviation know in the art (see, e.g., Lehninger, A. L., Biochemistry, second edition (Worth Publishers, Inc., New York, 1975), p. 72). A one or three-letter abbreviation is understood to indicate the L-amino acid, unless prefaced with “D-” to indicate the corresponding D-form. Moreover, the name of any acidic amino acid is understood to include its salt or ionized form. For example, L-aspartic acid is understood to also encompass L-aspartate.

Other abbreviations used herein include: “DHA” for a docosahexaenoic acid moiety; “Lip” for a lipoic acid moiety; “Palm” for a palmitic acid moiety (i.e., a palmitoyl group); “Ac” for an acetyl moiety; “Gga” for a glucose-3-O-glycolic acid moiety; “SOD” for super oxide dismutase; “CAT” for catalase; and “ROS” for reactive oxygen species. Still other abbreviations are indicated as needed elsewhere in the text.

“Hydrocarbon” refers to either branched or unbranched and saturated or unsaturated hydrocarbon chains. Preferred hydrocarbon chains found in some of the amino acid compounds described herein contain between 1 and 25. More preferred are hydrocarbon chains between 1 and 22 carbon atoms.

“Reactive oxygen species” or “ROS”, as understood and used herein, refers to highly reactive and toxic oxygen compounds that are generated in the course of normal electron transport system during respiration or that are generated in a disease or during treatment with certain therapeutic agents for a particular disorder. ROS include, but are not limited to, the superoxide anion (O₂.⁻), hydrogen peroxide (H₂O₂), singlet oxygen, lipid peroxides, and peroxynitrite.

“Free radical”, as understood and used herein, refers to any atom or any molecule or compound that possesses an odd (unpaired) electron. By this definition, the superoxide anion is also considered a negatively charged free radical. The free radicals of particular interest to this invention are highly reactive, highly oxidative molecules that are formed or generated during normal metabolism, in a diseased state, or during treatment with chemotherapeutic drugs. Such free radicals are highly reactive and capable of causing oxidative damage to molecules, cells and tissues. One of the most common and potentially destructive types of the free radicals other than the superoxide anion is a hydroxyl radical. Typically, the generation of ROS, such as superoxide anion or singlet oxygen, also leads to one or more other harmful free radicals as well. Accordingly, phrases such as “ROS and free radicals” or “ROS and other free radicals”, as understood and used herein, are meant to encompass any or all of the entire population of highly reactive, oxidative molecular species or compounds that may be generated in a particular metabolic state or condition of cells and tissues of interest (see, e.g., Somani et al, “Response of Antioxidant System To Physical and Chemical Stress,” In Oxidants, Antioxidants, and Free Radicals, chapter 6: 125-141 (Taylor & Francis, Washington, D.C., 1997)).

“Oxygen radical scavengers” or “lazaroids” are a class of compounds that have an ability to scavenge and detoxify ROS and free radicals. Vitamins A, C, E, and related antioxidant compounds, such as β-carotene and retinoids, are also members of this large class of compounds, as are antioxidative enzymes, such as SOD and CAT. In healthy individuals, sufficient levels of antioxidative enzymes and other lazaroids are present both intracellularly and extracellularly to efficiently scavenge sufficient amounts of ROS and free radicals to avoid significant oxidative damage to cells and tissues.

“Amino acid compound”, “single amino acid compound”, “single amino acid based compound”, and similar terms refer to any compound described herein that contains a single D- or L-amino acid, apart from any capping group as defined herein, and is capable of upregulating expression of a gene encoding an antioxidative enzyme, such as SOD and/or CAT. The single amino acid of a single amino acid compound described herein may be unmodified or a “derivative” of an amino acid, as defined herein.

An “amino terminal capping group” of an amino acid compound described herein is any chemical compound or moiety that is covalently linked or conjugated to the α amino group of an amino acid compound. The primary purpose of such an amino terminal capping group is to inhibit or prevent intermolecular polymerization and other undesirable reactions with other molecules, to promote transport of the compound across the blood-brain barrier, to provide an additional antioxidative activity, or to provide a combination of these properties. Thus, an amino acid compound of this invention that possesses an amino terminal capping group may exhibit other beneficial activities as compared with the uncapped amino acid, such as enhanced efficacy or reduced side effects. For example, several of the amino terminal capping groups used in the compounds described herein also possess antioxidative activity in their free state (e.g., lipoic acid) and thus, may improve or enhance the antioxidative activity of the uncapped amino acid. Examples of amino terminal capping groups that are useful in preparing amino acid compounds and compositions according to this invention include, but are not limited to, 1 to 6 lysine residues (SEQ ID NO:2), 1 to 6 arginine residues (SEQ ID NO:2), a mixture of arginine and lysine residues ranging from 2 to 6 residues (SEQ D NO:2), urethanes, urea compounds, a lipoic acid (“Lip”) or a palmitic acid moiety (i.e., palmitoyl group, “Palm”), glucose-3-O-glycolic acid moiety (“Gga”), or an acyl group that is covalently linked to the α amino group of the amino acid. Such acyl groups useful in the compositions of the invention may have a carbonyl group and a hydrocarbon chain that ranges from one carbon atom (e.g., as in an acetyl moiety) to up to 25 carbons (such as docosahexaenoic acid, “DHA”, which has a hydrocarbon chain that contains 22 carbons). Furthermore, the carbon chain of the acyl group may be saturated, as in a palmitic acid, or unsaturated. It should be understood that when an acid (such as DHA, Palm, or Lip) is present as an amino terminal capping group, the resultant amino acid compound is the condensed product of the uncapped amino acid and the acid.

A “carboxy terminal capping group” of an amino acid compound described herein is any chemical compound or moiety that is covalently linked or conjugated to the α carboxyl group of an amino acid of the amino acid compound. The primary purpose of such a carboxy terminal capping group is to inhibit or prevent intermolecular polymerization and other undesirable reactions with other molecules, to promote transport of the single amino acid compound across the blood-brain barrier, or to provide a combination of these properties. An amino acid compound of this invention possessing a carboxy terminal capping group may possess other beneficial activities as compared with the uncapped amino acid, such as enhanced efficacy, reduced side effects, enhanced hydrophilicity, enhanced hydrophobicity, or enhanced antioxidative activity, e.g., if the carboxy terminal capping moiety possesses a source of reducing potential, such as one or more sulfhydryl groups. Carboxy terminal capping groups that are particularly useful in the amino acid compounds described herein include primary or secondary amines that are linked by an amide bond to the α carboxyl group of the amino acid compound. Other carboxy terminal capping groups useful in the invention include aliphatic primary and secondary alcohols and aromatic phenolic derivatives, including flavenoids, with C1 to C26 carbon atoms, which form esters when linked to the α carboxyl group of an amino acid compound described herein.

A “derivative” of an amino acid refers to an amino acid that contains one or more chemical groups that are attached, preferably covalently, to the side chain of the unmodified amino acid residue. Preferred derivatives of amino acids of the invention contain chemical groups that do not adversely affect or destroy the activity of the amino acid compound to upregulate expression of a gene encoding an antioxidative enzyme, such as a gene encoding SOD and/or CAT.

“Natural source purified”, as understood and used herein, describes a composition of matter purified or extracted from an organism or collection of organisms occurring in nature or in a cultivated state that have not been altered genetically by in vitro recombinant nucleic acid technology, including but not limited to animals, any species of crops used for beverage and food, species of uncultivated plants growing in nature, species of plants developed from plant breeding, and microorganisms that have not been altered genetically by in vitro recombinant technology.

“Radiation”, as understood and used herein, means any type of propagating or emitted energy wave or energized particle, including electromagnetic radiation, ultraviolet radiation (UV), and other sunlight-induced radiation and radioactive radiation. The effects of such radiation may affect the surface or underlayers of the skin or may produce systemic damage at a remote site in the body.

“Upregulate” and “upregulation”, as understood and used herein, refer generally to an elevation in the level of expression of a gene in a cell or tissue. An elevation of gene expression is correlated with and detected herein by higher levels of expression of the gene's product, e.g., a transcript or a protein, so that the terms “upregulate” and “upregulation” may be properly applied to describe an elevation in the level of expression of a gene's product as well. Peptide compounds described herein are capable of upregulating expression of a gene(s) encoding the antioxidative enzyme superoxide dismutase (SOD) and/or catalase (CAT) beyond the levels normally found in cells or tissues that have not been treated (contacted) with the peptide compounds. Thus, an elevation in the level of SOD or CAT mRNA transcript; in SOD or CAT gene product (protein) synthesis; or in the level of SOD or CAT enzyme activity indicates an upregulation of expression of a gene(s) encoding an antioxidative enzyme. Expression of SOD and CAT genes can be detected by a variety of methods including, but not limited to, Northern blotting to detect mRNA transcripts encoding SOD and/or CAT, Western immunoblotting to detect the gene product, i.e., SOD and/or CAT protein, and standard assays for SOD or CAT enzymatic activities.

“Nutraceutical” and “dietary supplement”, as understood and used herein, are synonymous terms, which describe compositions that are prepared and marketed for sale as non-regulated, orally administered, sources of a nutrient and/or other compound that is purported to contain a property or activity that may provide a benefit to the health of an individual. A desirable component compound identified in a dietary supplement is referred to as a “nutrichemical”. Nutrichemicals may be present in only trace amounts and still be a desirable and marketable component of a dietary supplement. Commonly known nutrichemicals include trace metals, vitamins, enzymes that have an activity that is considered beneficial to the health of an individual, and compounds that upregulate such enzymes. Such enzymes include antioxidative enzymes, such as superoxide dismutase (SOD) and catalase (CAT), which counteract the harmful oxidative effects of reactive oxygen species (ROS) and other free radicals. Accordingly, one or more single amino acid compounds described herein that are endogenously present and/or added exogenously to a composition manufactured for sale as a dietary supplement is a nutrichemical of that dietary supplement.

Other terms will be evident as used in the following description.

Single Amino Acid Compounds and Compositions

The invention provides single amino acid compounds described herein for use in compositions and/or methods for upregulating expression of SOD and/or CAT in eukaryotic cells. Upregulating levels of SOD and/or CAT in cells or tissues provides an enhanced detoxification system to prevent, reduce, or eliminate the harmful oxidative activity of ROS and free radicals on cells and tissues. Preferred single amino acid compounds of this invention upregulate both SOD and CAT. The activity of the single amino acid compounds described herein to upregulate SOD and/or CAT may be measured in vitro, e.g., in tissue culture, or in vivo using any of number of available methods.

The invention also provides compositions comprising a single amino acid based compound having the formula:
R₁-Xaa-R₂,
wherein:

R₁ is absent or is an amino terminal capping group;
Xaa is any amino acid, or derivative thereof, that upregulates expression of a gene encoding an antioxidative enzyme;
R₂ is absent or is a carboxy terminal capping group; and wherein the single amino acid based compound upregulates expression of a gene encoding an antioxidative enzyme.

Preferably, Xaa in the above formula is an amino acid selected from the group consisting of L-aspartic acid, D-aspartic acid, L-asparagine, D-asparagine, L-glutamic acid, D-glutamic acid, L-glutamine, D-glutamine, and derivatives thereof, and is capable of upregulating expression of an antioxidative enzyme, such as SOD and/or CAT. L-aspartic acid is particularly preferred.

In a preferred embodiment, the invention provides a composition comprising a single amino acid based compound of the above formula, i.e., R₁-Xaa-R₂ (SEQ ID NO:1), wherein Xaa is L-aspartic acid, L-asparagine, or derivatives thereof, and wherein the single amino acid compound upregulates expression of a gene encoding an antioxidative enzyme. Most preferably, Xaa is L-aspartic acid.

Preferably, a gene(s) upregulated by an amino acid compound of the invention encodes an antioxidative enzyme(s), which is superoxide dismutase (SOD) and/or catalase (CAT).

The single amino acid compounds useful in the invention include the group of unmodified, uncapped amino acids consisting of L-aspartic acid, L-asparagine, D-aspartic acid, D-asparagine, L-glutamic acid, D-glutamic acid, and D-glutamine. More preferably, the single amino acid compound of the invention is L-aspartic acid or L-asparagine, and most preferably L-aspartic acid.

The single amino acid compounds described herein may contain a derivative of an amino acid, in which additional modifications have been made, such as linking, preferably covalently, a chemical group to the side chain of the amino acid residue, provided such modification does not destroy the desired activity of the amino acid compound to upregulate expression of an antioxidative enzyme.

The single amino acid compounds of the invention may contain an amino terminal capping group (“R₁” in the above formula) linked to the α amino group of the amino acid. Such capping groups may provide any of a variety of functions, including but not limited to, providing a means to prevent undesirable or to enable desirable polymerization with other molecules, including another sister single amino acid based compound, e.g., to form a dimer or other multimer form of a single amino acid based compound; providing a means to link the single amino acid based compound to a substrate, e.g., to a resin particle, membrane, surface of a well of a microtiter plate, and the like; or providing a means for promoting transport of the single amino acid compound across the blood-brain barrier (see, e.g., PCT publication WO 99/26620). This latter property is particularly important when a single amino acid compound is used to upregulate expression of a gene(s) for an antioxidative enzyme, such as SOD and/or CAT, in brain tissue and parts of the central nervous system. Amino terminal capping groups that promote transport across the blood-brain barrier may also prevent the single amino acid compound from undesired reactions with other molecules.

Preferred amino terminal capping groups include a lipoic acid (“Lip”) moiety, which can be attached by an amide linkage to the α-amino group of an amino acid. Lipoic acid in its free form possesses independent antioxidative activity and, thus, may further enhance the antioxidative activity of the single amino acid compounds of this invention when used as an amino terminal capping group. An amino terminally linked lipoic acid moiety may be in its reduced form where it contains two sulfhydryl groups or in its oxidized form in which the sulfhydryl groups are oxidized and form an intramolecular disulfide bond and, thereby, a heterocyclic ring structure. Another amino terminal capping group useful in preparing single amino acid compounds of the invention is a glucose-3-O-glycolic acid moiety (“Gga”), which can be attached in an amide linkage to the α-amino group of the amino acid of a single amino acid compound. The glucose moiety may also contain further modifications, such as an alkoxy group replacing one or more of the hydroxyl groups on the glucose moiety.

Another example of an amino terminal capping group useful in the single amino acid compounds described herein is an acyl group, which can be attached in an amide linkage to the α-amino group of the amino acid residue of the single amino acid compound. The acyl group has a carbonyl group linked to a saturated or unsaturated (mono- or polyunsaturated), branched or unbranched, hydrocarbon chain of preferably 1 to 25 carbon atoms in length, and more preferably, the hydrocarbon chain of the acyl group is 1 to 22 carbon atoms in length, as in docosahexaenoic acid (DHA). The acyl group preferably is an acetyl group or a fatty acyl group. A fatty acid used as the fatty acyl amino terminal capping group may contain a hydrocarbon chain that is saturated or unsaturated and that is either branched or unbranched. Preferably the hydrocarbon chain of the fatty acid is 1 to 25 carbon atoms in length, and more preferably the length of the hydrocarbon chain is 1-22 carbon atoms in length. For example, fatty acids that are useful as fatty acyl amino terminal capping groups for the amino acid compounds of this invention include, but are not limited to: caprylic acid (C8:0), capric acid (C10:0), lauric acid (C12:0), myristic acid (C14:0), palmitic acid (“Palm”) (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22:1), docosatetraenoic acid (C22:4-6), docosapentaenoic acid (C22:5-6), docosapentaenoic acid (C22:5-3), docosahexaenoic acid (“DHA”) (C22:6-3), and nervonic acid (C24:1-9). Particularly preferred fatty acids used as acyl amino terminal capping groups for the single amino acid compounds described herein are palmitic acid (Palm) and docosahexaenoic acid (DHA). DHA and various other fatty acid moieties appear to promote transport of molecules to which they are linked across the blood-barrier (see, e.g., PCT publication WO 99/40112 and PCT publication WO 99/26620). Accordingly, such fatty acyl moieties are particularly preferred when a single amino acid compound described herein will be administered to counteract the oxidative effects of ROS and free radicals in brain tissue and/or other parts of the central nervous system.

In addition, in certain cases the amino terminal capping group may be a lysine residue or a polylysine peptide, preferably where the polylysine peptide consists of two, three, four, five or six lysine residues (SEQ ID NO:2), which can prevent cyclization, crosslinking, or polymerization of the single amino acid compound with itself or other molecules. Longer polylysine peptides conceivably may also be used. Another amino terminal capping group that may be used in the single amino acid compounds described herein is an arginine residue or a polyarginine peptide, preferably where the polyarginine peptide consists of two, three, four, five, or six arginine residues (SEQ ID NO:2), although longer polyarginine peptides may also be used. An amino terminal capping group of the single amino acid compounds described herein may also be a peptide containing both lysine and arginine, preferably where the lysine and arginine containing peptide is two, three, four, five, or six residue combinations of the two amino acids in any order (SEQ ID NO:2), although longer peptides that contain lysine and arginine conceivably may also be used (i.e., multimers of SEQ ID NO:2). Lysine and arginine containing peptides used as amino terminal capping groups in the single amino acid compounds described herein may be conveniently incorporated into whatever process is used to synthesize the amino acid compounds to yield the final product compound containing the amino terminal capping group.

The single amino acid compounds useful in the compositions and methods of the invention may contain a carboxy terminal capping group (“R₂” is the above formula). The primary purpose of this group is to prevent undesired reaction with other molecules as well as intermolecular crosslinking or polymerization. However, as noted above, a carboxy terminal capping group may provide additional benefits to the single amino acid compound, such as enhanced efficacy, reduced side effects, enhanced antioxidative activity, and/or other desirable biochemical properties. An example of such a useful carboxy terminal capping group is a primary or secondary amine in an amide linkage to the α carboxyl group of the amino acid residue of the compound. Such amines may be added to the α carboxyl group of the amino acid using standard amidation chemistry.

As noted above, single amino acid compounds described herein may contain the L or the D form of an amino acid residue as long as the single amino acid compound upregulates expression of an antioxidative enzyme, such as SOD and/or CAT. Use of a D-amino acid in place of the corresponding L-amino acid may advantageously provide additional stability to an amino acid compound, especially in vivo. Other conventional factors such as toxicity and other side effects must also be considered when selecting particular amino acids or isomeric forms.

The amino acid compounds described herein may be produced using standard methods or obtained from a commercial source. Both L and D forms of amino acids are commercially available or may be purified from various sources. Addition of capping groups to an amino acid may be carried out by standard chemical reactions, e.g., for acylation, amidation, and condensations. If the capping group consists of one or more amino acid, such amino acids may be linked to the single amino acid of a compound by standard peptide bond formation or by direct synthesis of the peptide formed between the single amino acid and the amino acid residues of the capping group by synthetic methods that are well-known by those of skill in the art (see, Stewart et al., Solid-Phase Peptide Synthesis (W. H. Freeman Co., San Francisco 1989); Merrifield, J. Am. Chem. Soc., 85:2149-2154 (1963); Bodanszky and Bodanszky, The Practice of Peptide Synthesis (Springer-Verlag, New York 1984), incorporated herein by reference).

Single amino acid compounds useful in the compositions and methods of the invention may also be prepared and used in a salt form. Typically, a salt form of an amino acid compound will exist by adjusting the pH of a composition comprising the amino acid compound with an acid or base in the presence of one or more ions that serve as counter ions to the net ionic charge of the amino acid compound at the particular pH. Various salt forms of the amino acid compounds described herein may also be formed or interchanged by any of the various methods known in the art, e.g., by using various ion exchange chromatography methods. Cationic counter ions that may be used in the compositions described herein include, but are not limited to, amines, such as ammonium ion; metal ions, especially monovalent, divalent, or trivalent ions of alkali metals (e.g., sodium, potassium, lithium, cesium), alkaline earth metals (e.g., calcium, magnesium, barium), transition metals (e.g., iron, manganese, zinc, cadmium, molybdenum), other metals (e.g., aluminum); and combinations thereof. Anionic counter ions that may be used in the compositions described herein include, but are not limited to, chloride, fluoride, acetate, trifluoroacetate, phosphate, sulfate, carbonate, citrate, ascorbate, sorbate, glutarate, ketoglutarate, and combinations thereof. Trifluoroacetate salts of amino acid compounds described herein are typically formed during purification in trifluoroacetic acid buffers using high-performance liquid chromatography (HPLC). While generally not suited for in vivo use, trifluoroacetate salt forms of a single amino acid compound described herein may be conveniently used in various in vitro cell culture studies or assays performed to test the activity or efficacy of the amino acid compound. The amino acid compound may then be converted from the trifluoroacetate salt (e.g., by ion exchange methods) to a less toxic salt or synthesized and produced as a salt form that is acceptable for pharmaceutical or dietary supplement (nutraceutical) compositions.

A single amino acid compound useful in the invention is preferably obtained in a purified form, acceptable for administration to an individual as a pharmaceutical composition or nutraceutical. For purification purposes, there are many standard methods that may be employed, including standard chromatographic techniques and various methods of reversed-phase high-pressure liquid chromatography (HPLC). An amino acid compound that produces a single peak is at least 95% of the input material on an HPLC column is preferred. Even more preferable is a compound that produces a single peak that is (in order of increasing preference) at least 97%, at least 98%, at least 99% or even at least 99.5% of the input material on an HPLC column.

In order to ensure the identity of a single amino acid compound, analysis of the compound's composition may be determined by any of a variety of analytical methods known in the art. Such composition analysis may be conducted using tests for a particular amino acid and high resolution mass spectrometry. Thin-layer chromatographic (TLC) methods may also be used to authenticate a single amino acid compound of the invention.

The single amino acid compounds described herein are useful in the compositions and methods of the invention to upregulate the expression of a gene encoding an antioxidative enzyme, such as SOD and/or CAT, and thereby generate antioxidative activity to counteract the undesirable and destructive oxidative activity of ROS and free radicals, e.g., as generated in the aging process (senescence), disease, and various drug treatments.

Single amino acid compounds that upregulate a gene encoding an antioxidative enzyme and that are useful in compositions and methods of the invention may include, but are not limited to, L-aspartic acid, D-aspartic acid, L-asparagine, D-asparagine, L-glutamic acid, D-glutamic acid, L-glutamine, D-glutamine, and derivatives thereof. Particularly preferred are L-aspartic acid and derivatives thereof.

Biological and Biochemical Activities

The single amino acid compounds useful in the compositions and methods of the invention have the ability to upregulate expression of a gene encoding an antioxidative enzyme, such as SOD and/or CAT, in cells and tissues, especially mammalian cells, provided the cells contain a functional gene(s) encoding such an enzyme(s). A functional gene is one, which not only encodes a particular enzyme, but also provides the necessary genetic information within and without the coding sequence so that transcription of the gene can occur and so that the mRNA transcript can be translated into a functioning gene product.

Certain preferred single amino acid compounds described herein are able to upregulate expression of both SOD and CAT, again assuming that functional genes for both enzymes are present in the cells of interest. Advantageously, upregulation of SOD and CAT together provide enhanced efficacy in detoxifying undesired ROS and free radicals. Without wishing to be bound by any particular mechanism or theory, when the level of SOD protein increases as a result of upregulation of SOD gene expression, it is believed that superior antioxidative efficacy is achieved when there is also an increase in CAT levels. Upregulation of a gene for CAT increases the capacity to neutralize and detoxify the additional hydrogen peroxide and other ROS or free radicals that can be generated by enhanced SOD activity. The single amino acid compounds described herein having both SOD and CAT upregulation activity provide cells and tissues with a full complement of enhanced antioxidative enzyme activity to detoxify ROS and free radicals. For example, contacting mammalian cells in tissue culture with a single amino acid compound described herein having both SOD and CAT upregulation activity typically results in at least about a 2-fold, and in increasing order of preference, at least about a 3-fold, 4-fold, and 6 to 8-fold increase in the levels of expression of SOD and CAT protein, as detected by immunoblotting and compared to untreated cells. Such increase in levels of SOD and CAT gene expression provides a cell with a significantly enhanced capability for detoxifying ROS and free radicals without adverse effects.

Expression of genes encoding SOD and CAT can be measured by a variety of methods. Standard enzymatic assays are available to detect levels of SOD and CAT in cell and tissue extracts or biological fluids (Fridovich, Adv. Enzymol., 41:35-97 (1974); Beyer & Fridovich, Anal. Biochem., 161:559-566 (1987)). In addition, antibodies to SOD and CAT are available or readily made. Using such antibodies specific for each protein, standard immunoblots (e.g., Western blots) and other immunological techniques can be used to measure levels of SOD and CAT in various mixtures, cell extracts, or other sample of biological material. Provided there is no evidence of a defect in the translation machinery of the cells of interest, the levels of expression of genes encoding SOD and CAT can also be measured by detecting levels of mRNA transcripts using standard Northern blot or standard polymerase chain reaction (PCR) methods for measuring specific mRNA species (e.g., RT-PCR).

Therapeutic and Prophylactic Applications

The single amino acid based compounds useful in the invention upregulate expression of a gene(s) encoding an antioxidative enzyme(s), such as SOD and/or CAT, in cells and tissues of animals, including humans and other mammals. Preferably, the amino acid based compounds of this invention upregulate expression of both SOD and CAT. As noted above, SOD and CAT comprise components of the body's major enzymatic antioxidative activities that are able to detoxify ROS and free radicals by reducing such molecules to less reactive and less harmful compounds. The contribution of ROS and other free radicals to the progression of various disease states and side effects of drugs is now well known.

For example, elevated levels of ROS and free radicals are known to be generated in cells and tissues during reperfusion after an ischemic event. Such increased levels of ROS and free radicals can cause considerable damage to an already stressed or debilitated organ or tissue. The single amino acid compounds of this invention, which upregulate SOD and/or CAT, may be used to treat reperfusion injuries that occur in diseases and conditions such as stroke, heart attack, or renal disease and kidney transplants. If the ischemic event has already occurred as in stroke and heart attack, a single amino acid compound described herein may be administered to the individual to detoxify the elevated ROS and free radicals already present in the blood and affected tissue or organ. Alternatively, if the ischemic event is anticipated as in organ transplantation, then single amino acid compounds described herein may be administered prophylactically, prior to the operation or ischemic event.

Although a major application is in the treatment of ischemia-reperfusion injury, the single amino acid compounds described herein may be used to treat any disease or condition associated with undesirable levels of ROS and free radicals or to prevent any disease, disorder or condition caused by undesirable levels of ROS and free radicals. According to the invention, the single amino acid compounds described herein may also be administered to provide a therapeutic or prophylactic treatment of elevated ROS and other free radicals associated with a variety of other diseases and conditions, including, but not limited to, oxygen toxicity in premature infants, burns and physical trauma to tissues and organs, septic shock, polytraumatous shock, head trauma, brain trauma, spinal cord injuries, Parkinson's disease, amyotrophic lateral sclerosis (ALS), Alzheimer's disease, age-related elevation of ROS and free radicals, senility, ulcerative colitis, human leukemia and other cancers, Down syndrome, arthritis, macular degeneration, schizophrenia, epilepsy, radiation damage (including UV-induced skin damage), and drug-induced increase in ROS and free radicals.

A progressive rise of oxidative stress due to the formation of ROS and free radicals occurs during aging (see, e.g., Mecocci, P. et al., Free Radic. Biol. Med., 28: 1243-1248 (2000)). This has been detected by finding an increase in the formation of lipid peroxidates in rat tissues (Erdincler, D. S., et al., Clin. Chim. Acta, 265: 77-84 (1997)) and blood cells in elderly human patients (Congi, F., et al., Presse. Med., 24: 1115-1118 (1995)). A recent review (Niki, E., Intern. Med., 39: 324-326 (2000)) reported that increased tissue damage by ROS and free radicals could be attributed to decreased levels of the antioxidative enzymes SOD and CAT that occurs during aging. For example, transgenic animals, generated by inserting extra SOD genes into the genome of mice were found to have decreased levels of ROS and free radical damage. Such animals also had an extended life span. More recent evidence indicated that administration of a small manganese porphyrin compound, which mimics SOD activity, led to a 44% extension of life span of the nematode worm Caenorhabditis elegans (S. Melow, et al., Science, 289: 1567-1569 (2000)). Accordingly, the single amino acid based compounds described herein, which are able to upregulate expression of SOD and/or CAT genes to produce increased levels of antioxidative enzymes, are also well suited for use in methods of preventing and/or counteracting increased tissue damage and decreased life expectancy due to elevated levels of ROS and free radicals that accompany the aging process.

A variety of drugs in current therapeutic use produce tissue-specific toxic side effects that are correlated with an elevation in the levels of ROS and other free radicals. Such drugs include neuroleptics, antibiotics, analgesics, and other classes of drugs. The tissues affected by such drug-induced toxicities can include one or more of the major organs and tissues, such as brain, heart, lungs, liver, kidney, and blood. Accordingly, in one aspect of the invention, a single amino acid compound described herein may be administered to an individual prior to, simultaneously with, or after administration of a drug that is known or suspected of increasing ROS and free radicals.

Pharmaceutical Applications

Pharmaceutical compositions of this invention comprise a single amino acid compound described herein, or pharmaceutically acceptable salts thereof, with any pharmaceutically acceptable carrier, ingredient, excipient, adjuvant, or vehicle.

Pharmaceutical compositions of this invention can be administered to mammals, including humans, in a manner similar to other therapeutic, prophylactic, or diagnostic agents, and especially therapeutic hormone peptides. The dosage to be administered, and the mode of administration will depend on a variety of factors including age, weight, sex, condition of the patient, and genetic factors, and will ultimately be decided by the attending physician or veterinarian. In general, dosage required for diagnostic sensitivity or therapeutic efficacy will range from about 0.001 to 25.0 μg/kg of host body mass.

Pharmaceutically acceptable salts of the single amino acid compounds of this invention include, e.g., those derived from pharmaceutically acceptable inorganic and organic acids and bases. Examples of suitable acids include hydrochloric, hydrobromic, sulfuric, nitric, perchloric, fumaric, maleic, malic, pamoic, phosphoric, glycolic, lactic, salicylic, succinic, toluene-p-sulfonic, tartaric, acetic, citric, methanesulfonic, formic, benzoic, malonic, naphthalene-2-sulfonic, tannic, carboxymethyl cellulose, polylactic, polyglycolic, and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N—(C₁₋₄ alkyl)₄⁺ salts.

This invention also envisions the “quaternization” of any basic nitrogen-containing groups of a single amino acid compound disclosed herein, provided such quaternization does not destroy the ability of the compound to upregulate expression of genes encoding SOD and CAT. Even more preferred is the quaternized single amino acid compound in which the α carboxyl group is converted to an amide to prevent the carboxyl group from reacting with any free amino groups present either on other molecules or within the compound itself. Any basic nitrogen can be quaternized with any agent known to those of ordinary skill in the art including, e.g., lower alkyl halides, such as methyl, ethyl, propyl, or butyl chloride, bromides, and iodides; dialkyl sulfates, including dimethyl, diethyl, dibutyl and diamyl sulfates; long chain halides such as decyl, lauryl, myristyl and stearyl chlorides, bromides and iodides; and aralkyl halides, including benzyl and phenethyl bromides. Water or oil-soluble or dispersible products may be obtained by such quaternization or using acids such as acetic acid and hydrochloric acid.

It should be understood that the single amino acid compounds of this invention may be modified by appropriate functionalities to enhance selective biological properties, and in particular the ability to upregulate expression of SOD and/or CAT. Such modifications are known in the art and include those, which increase the ability of the single amino acid compound to penetrate or be transported into a given biological system (e.g., brain, central nervous system, blood, lymphatic system), increase oral availability, increase solubility to allow administration by injection, alter metabolism of the amino acid compound, and alter the rate of excretion of the single amino acid compound. In addition, a single amino acid compound of the invention may be altered to a pro-drug form such that the desired amino acid compound is created in the body of the patient as the result of the action of metabolic or other biochemical processes on the pro-drug. Such pro-drug forms typically demonstrate little or no activity in in vitro assays. Some examples of pro-drug forms may include ketal, acetal, oxime, and hydrazone forms of compounds which contain ketone or aldehyde groups. Other examples of pro-drug forms include the hemi-ketal, hemi-acetal, acyloxy ketal, acyloxy acetal, ketal, and acetal forms.

Pharmaceutically acceptable carriers, adjuvants, and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene -block polymers, polyethylene glycol, and wool fat.

The pharmaceutical compositions of this invention may be administered by a variety of routes or modes. These include, but are not limited to, parenteral, oral, intratracheal, sublingual, pulmonary, topical, rectal, nasal, buccal, sublingual, vaginal, or via an implanted reservoir. Oral administration is preferred. Implanted reservoirs may function by mechanical, osmotic, or other means. The term “parenteral”, as understood and used herein, includes intravenous, intracranial, intraperitoneal, paravertebral, periarticular, periostal, subcutaneous, intracutaneous, intra-arterial, intramuscular, intra-articular, intrasynovial, intrasternal, intrathecal, and intralesional injection or infusion techniques. Such compositions are preferably formulated for parenteral administration, and most preferably for intravenous, intracranial, or intraarterial administration. Generally, and particularly when administration is intravenous or intra-arterial, pharmaceutical compositions may be given as a bolus, as two or more doses separated in time, or as a constant or non-linear flow infusion.

The pharmaceutical compositions may be in the form of a sterile injectable preparation, e.g., as a sterile injectable aqueous or oleaginous suspension. This suspension may be formulated according to techniques known in the art using suitable dispersing or wetting agents (such as, e.g., Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as those described in Pharmacoplia Halselica.

The pharmaceutical compositions of this invention may be orally administered in any orally acceptable dosage form including, but not limited to, capsules, tablets, caplets, pills, aqueous or oleaginous suspensions and solutions, syrups, or elixirs. In the case of tablets for oral use, carriers, which are commonly used include lactose and corn starch. Lubricating agents, such as magnesium stearate, are also typically added. For oral administration in a capsule form, useful diluents include lactose and dried cornstarch. Capsules, tablets, pills, and caplets may be formulated for delayed or sustained release.

When aqueous suspensions are to be administered orally, a single amino acid compound of the invention is advantageously combined with emulsifying and/or suspending agents. If desired, certain sweetening and/or flavoring and/or coloring agents may be added. Formulations for oral administration may contain 10%-95% active ingredient, preferably 25%-70%. Preferably, a pharmaceutical composition for oral administration provides a single amino acid compound of the invention in a mixture that prevents or inhibits hydrolysis of the single amino acid compound by the digestive system, but allows absorption into the blood stream.

The pharmaceutical compositions of this invention may also be administered in the form of suppositories for vaginal or rectal administration. These compositions can be prepared by mixing a compound of this invention with a suitable non-irritating excipient, which is solid at room temperature but liquid at body temperature and therefore will melt in relevant body space to release the active components. Such materials include, but are not limited to, cocoa butter, beeswax and polyethylene glycols. Formulations for administration by suppository may contain 0.5%-10% active ingredient, preferably 1%-2%.

Topical administration of the pharmaceutical compositions of this invention may be useful when the desired treatment involves areas or organs accessible by topical application, such as in wounds or during surgery. For application topically, the pharmaceutical composition may be formulated with a suitable ointment containing the active components suspended or dissolved in a carrier. Carriers for topical administration of the single amino acid compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing a single amino acid compound suspended or dissolved in a pharmaceutically suitable carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical composition may be formulated for topical or other application as a jelly, gel, or emollient, where appropriate. The pharmaceutical compositions of this invention may also be topically applied to the lower intestinal tract by rectal suppository formulation or in a suitable enema formulation. Topical administration may also be accomplished via transdermal patches. This may be useful for maintaining a healthy skin tissue and restoring oxidative skin damage (e.g., UV- or radiation-induced skin damage).

The pharmaceutical compositions of this invention may be administered nasally, in which case absorption may occur via the mucus membranes of the nose, or inhalation into the lungs. Such modes of administration typically require that the composition be provided in the form of a powder, solution, or liquid suspension, which is then mixed with a gas (e.g., air, oxygen, nitrogen, etc., or combinations thereof) so as to generate an aerosol or suspension of droplets or particles. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Pharmaceutical compositions of the invention may be packaged in a variety of ways appropriate to the dosage form and mode of administration. These include but are not limited to vials, bottles, cans, packets, ampoules, cartons, flexible containers, inhalers, and nebulizers. Such compositions may be packaged for single or multiple administrations from the same container. Kits, of one or more doses, may be provided containing both the composition in dry powder or lyophilized form, as well an appropriate diluent, which are to be combined shortly before administration. The pharmaceutical composition may also be packaged in single use pre-filled syringes, or in cartridges for auto-injectors and needleless jet injectors.

Multi-use packaging may require the addition of antimicrobial agents such as phenol, benzyl alcohol, meta-cresol, methyl paraben, propyl paraben, benzalconium chloride, and benzethonium chloride, at concentrations that will prevent the growth of bacteria, fungi, and the like, but be non-toxic when administered to an individual.

Consistent with good manufacturing practices, which are in current use in the pharmaceutical industry and which are well known to the skilled practitioner, all components contacting or comprising the pharmaceutical agent must be sterile and periodically tested for sterility in accordance with industry norms. Methods for sterilization include ultrafiltration, autoclaving, dry and wet heating, exposure to gases such as ethylene oxide, exposure to liquids, such as oxidizing agents, including sodium hypochlorite (bleach), exposure to high energy electromagnetic radiation, such as ultraviolet light, x-rays or gamma rays, and exposure to ionizing radiation. Choice of method of sterilization will be made by the skilled practitioner with the goal of effecting the most efficient sterilization that does not significantly alter a desired biological function, i.e., the ability to upregulate SOD or CAT, of the pharmaceutical agent in question. Ultrafiltration is a preferred method of sterilization for pharmaceutical compositions that are aqueous solutions or suspensions.

Details concerning dosages, dosage forms, modes of administration, composition and the like are further discussed in a standard pharmaceutical text, such as Remington's Pharmaceutical Sciences, 18th ed., Alfonso R. Gennaro, ed. (Mack Publishing Co., Easton, Pa. 1990), which is hereby incorporated by reference.

As is well known in the art, structure and biological function of amino acids are sensitive to chemical and physical environmental conditions such as temperature, pH, oxidizing and reducing agents, freezing, shaking and shear stress. Due to this inherent susceptibility to degradation, it is necessary to ensure that the biological activity of a single amino acid compound of the invention when present in a pharmaceutical composition be preserved during the time that the composition is manufactured, packaged, distributed, stored, prepared and administered by a competent practitioner.

Natural Source Purified Compositions and Dietary Supplements

The invention also provides compositions and methods of making such compositions for use as dietary supplements (also referred to as “nutraceuticals”) comprising a natural source purified composition obtained from an organism (i.e., animal, plant, or microorganism), which purified composition contains an endogenous single amino acid or a single amino acid compounds described herein, which upregulates expression of one or more genes encoding an antioxidative enzyme, such as SOD and/or CAT in cells or tissues. Amino acid compounds of the invention may be obtained in highly purified form from some natural sources. The level of such amino acid compounds in natural materials may be quite low or even present in only a trace amount, accordingly, to obtain useful quantities, the single amino acid compounds described herein may be made synthetically. Accordingly, dietary supplement compositions of the invention may further comprise an exogenously provided amino acid or a single amino acid compound described herein that upregulates expression of one or more genes encoding an antioxidative enzyme, such as SOD and/or CAT. Preferred natural sources of purified compositions used in making dietary supplements of the invention include plants, animals, and microorganisms.

Dietary supplement formulations of the invention may comprise a natural source purified composition comprising an endogenous single amino acid compound described herein. Other dietary supplement formulations of the invention are compositions which comprise a natural source purified composition that contains an endogenous amino acid or an single amino acid compound, which is capable of upregulating expression of SOD and/or CAT, and that is combined with one or more exogenously provided single amino acid compounds described herein. An advantage of this latter type of formulation is that a sufficient amount of an exogenously provided single amino acid compound described herein may be combined with the natural source purified composition to form a dietary supplement composition that produces a desirable level or range of levels of upregulated antioxidative enzymes in an individual that takes or is administered the dietary supplement. Accordingly, dietary supplement compositions of the invention may contain one or more different single amino acid compounds described herein as an endogenous compound from a natural source purified composition as well as, if so formulated, an exogenously provided single amino acid compound described herein.

Natural source purified compositions can be assayed for the presence of one or more single amino acid compounds, and the activity to upregulate expression of a gene encoding SOD and/or CAT assayed in vitro or in vivo in mammalian cells by any of the various methods described herein or their equivalents. Such analysis provides the information that enables the consistent manufacture of standardized lots of an oral dietary supplement product, which contains an appropriate amount of a single amino acid compound to provide the same or substantially the same lot to lot antioxidative activity to an individual who takes the supplement. The ability to consistently manufacture and deliver for sale lots of the same oral supplement product having a standardized amount of an ingredient of interest is highly desired in the dietary supplements market where product consistency can play a critical role in establishing consumer confidence and patronage for a particular product.

Additional aspects of the invention will be further understood and illustrated in the following examples. The specific parameters included in the following examples are intended to illustrate the practice of the invention and its various features, and they are not presented to in any way limit the scope of the invention.

### EXAMPLES

### Example 1

### Effect of L-aspartic Acid on Primary Rat Cortical Cultures

Primary rat cortical cultures were obtained by growing newborn rat brain cortical cells in Delbecco's modified Eagle medium supplemented with 100 units/ml of penicillin G, 100 μg/ml of streptomycin, and 10% fetal calf serum. The cells were isolated from the E-21 cortex of rat brain, plated at a density of 1×10⁵ per ml and grown to confluence within four to five days in an atmosphere containing air and 5% CO₂ at 37° C. as described in Cornell-Bell et al., Science, 247: 470-473 (1990) and Cell Calcium, 12: 185-204 (1991). Cultures were grown in 20 ml flasks as a monolayer and then exposed to various concentrations of L-aspartic acid for studies of the effect on upregulation of the gene for SOD. Cultures of the rat brain cortical cells were incubated with 0.00, 0.01, 0.13, 1.30, and 13.30 μg/ml L-aspartic acid for durations of 5 hours. Control cultures were treated in the same manner, but were not incubated with L-aspartic acid.

Cytoplasmic proteins were isolated according to published methods (Adams et al., J. Leukoc. Biol., 62: 865-873 (1997)). The cell cultures were washed once in phosphate buffer saline (PBS) containing 20 mM EDTA and then suspended in 250 μl of freshly prepared lysis buffer (20 mM Hepes, pH 7.9, 10 mM KCl, 300 mM NaCl, 1 mM MgCl₂, 0.1% Triton X-100 nonionic detergent, 20% glycerol, 0.5 mM dithiothreitol (DTT), freshly supplemented with inhibitors as described in Adams et al., J. Biol. Chem., 77: 221-233 (2000)). The suspensions were then incubated for at least 10 minutes on ice to lyse cells and then centrifuged (14,000×g for 5 minutes at 4° C.) to pellet cell debris. The supernatant cytoplasmic fractions were removed and stored as aliquots at −80° C. for analysis. The protein concentrations of the cytoplasmic fraction varied within 2-6 μg/μl.

The cytoplasmic proteins were separated by SDS-PAGE using 5 μg/lane on the gels for analysis by Western immunoblots. The gels were processed for Western immunoblots basically as described by Adams et al. (General Cellular Biochemistry, 77: 221-233 (2000)) to measure upregulation of SOD. SOD expression was detected in the Western blots using anti-SOD rabbit polyclonal antibody (Rockland, Inc., Gilbertville, Pa.). The Western blots were also analyzed by laser densitometry to quantify SOD protein upregulation. The control in these experiments was an identical culture flask, which was treated only with vehicle (i.e., buffer, no L-aspartic acid). The results are shown in the table below, wherein upregulation of SOD and CAT is expressed as a fold increase relative to the results for untreated control cultures.

TABLE 1
Effect of Aspartic Acid on Primary Rat Brain Cortical Cultures

| | |
|---|---|
| | Upregulation |
| Dose | (fold increase) |

| (μg/ml) | SOD | CAT |
|---|---|---|
| 0.00 | 1.0 | 1.0 |
| 0.01 | 1.0 | 1.0 |
| 0.13 | 1.6 | 1.0 |
| 1.30 | 2.1 | 2.3 |
| 13.30 | 3.5 | 3.7 |

Aspartic acid at low doses of up to 10 ng/ml in culture had no effect on SOD upregulation or any aspects of tissue culture properties. However, if the level of L-aspartic acid is raised to 130 ng/ml, there is upregulation of SOD as shown in Table 1. When the concentration is raised by a factor of 10 to 1.3 μg/ml, then both SOD and CAT are upregulated. At a concentration of 13.3 μg/ml there was substantial increase of SOD as measured by the Western blot analytical methods.

### Example 2

### In Vivo Pharmacological Activity of L-aspartic Acid

In vivo experiments were carried out in Sprague-Dawley rats (300-325 g) with solutions of L-aspartic acid. The animals were injected intravenously (iv) via the tail vein with L-aspartic acid. Each animal received one injection of L-aspartic acid in normal saline at total dose equivalent of 0.00, 0.75, 1.50, 3.00, and 6.00 mg L-aspartic acid/kg body weight or orally by gavage at a dose equivalent of 0.00 or 60.0 mg/kg body weight. The animals were sacrificed by decapitation at 6 hours post injection and dissected to isolate the brain organs, which were frozen at −70° C. for subsequent analysis by Western immunoblots.

Each brain tissue was thawed and homogenized in a Down's homogenizer using ten volumes of homogenizer buffer (see, Adams et al., General Cellular Biochemistry, 77: 221-233 (2000); buffer as described in Adams et al., J. Leukoc. Biol., 62: 865-875 (1967)) to obtain a crude cytoplasmic fraction. The brain tissue homogenates were centrifuged (14,000×g for 5 minutes at 4° C.) to yield the supernatant purified cytoplasmic protein fractions for Western immunoblot analysis as described in Adams et al. (J. Cell. Biochem., 77: 221-233 (2000)). A 10 μg sample of each protein fraction was then separated by SDS polyacrylamide gel electrophoresis (SDS-PAGE) and analyzed for SOD and CAT content by Western blot assay as above.

Control for measurement of unstimulated levels of SOD were obtained from vehicle-only (i.e., no L aspartic acid), injected or gavaged rats that were sacrificed at 6 hours post injection. Both had essentially the same unstimulated level of SOD. Standard quantities of each cytoplasmic fraction (10 μg) were loaded on a lane of a gel for electrophoretic separation and Western immunoblot analysis (Adams et al., J. Cell. Biochem., 77: 221-233 (2000)). The stained gels were photographed and scanned by laser densitometry to quantify intensities in comparison to enzyme levels for control vehicle treated rats.

The results are shown in Table 2, below. The data are expressed as a fold increase relative to control animals that received vehicle only. Intravenous (i.v.) injections were administered as 0.3 ml in normal saline during five minutes. Oral doses were administered as a solution in 1 ml saline by gavage.

TABLE 2
In Vivo Studies of the Effect of Aspartic Acid on Upregulation of SOD
and CAT Genes in Rat Brain

| | |
|---|---|
| | Fold |
| Dose | Upregulation of |

| (mg/kg) | Delivery Method | SOD | CAT |
|---|---|---|---|
| 0.00 | i.v. | 1.0 | 1.0 |
| 0.75 | i.v. | 1.0 | 1.0 |
| 1.50 | i.v. | 1.0 | 1.0 |
| 3.00 | i.v. | 1.3 | 1.2 |
| 6.00 | i.v. | 1.3 | 2.5 |
| 0.00 | oral | 1.0 | 1.0 |
| 60.00 | oral | 2.9 | 3.5 |

In vivo, doses of 3 mg/Kg must be injected i.v., before upregulation of SOD and CAT is observed. At 6 mg/Kg, upregulation of CAT became higher than SOD. The compound is also active orally, but in that case, a dose of 60 mg/Kg must be used in the rat to see an effect. At this level there is no toxic effect. There was, however, a mild increase in urination. Otherwise, the animals behaved normally.

Other variations and embodiments of the invention described herein will now be apparent to those of ordinary skill in the art without departing from the scope of the invention.

All patents, applications, and publications cited in the above text are incorporated herein by reference.

1. A composition comprising a single amino acid based compound having the formula:
R₁-Xaa-R₂ (SEQ ID NO:1),
wherein:
R₁ is absent or is an amino terminal capping group;
Xaa is any amino acid, or derivative thereof, that upregulates expression of a gene encoding an antioxidative enzyme;
R₂ is absent or is a carboxy terminal capping group; and
wherein said single amino acid based compound upregulates expression of a gene encoding an antioxidative enzyme.

2. The composition according to claim 1, wherein said antioxidative enzyme is superoxide dismutase (SOD) or catalase (CAT).

3. The composition according to claim 1, wherein said Xaa is selected from the group consisting of L-aspartic acid, D-aspartic acid, L-asparagine, D-asparagine, L-glutamic acid, D-glutamic acid, L-glutamine, D-glutamine, and derivatives thereof.

4. The composition according to claim 3, wherein Xaa is L-aspartic acid, L-asparagine, or derivatives thereof.

5. The composition according to any of claims 1-4, wherein R₁, when present, is selected from the group consisting of: lipoic acid moiety (Lip); a glucose-3-O-glycolic acid (Gga) moiety; 1 to 6 lysines (SEQ ID NO:2); 1 to 6 arginine residues (SEQ ID NO:2); a lysine and arginine containing peptide of 2-6 amino acid residues (SEQ ID NO:2); an acyl group having the formula R₃—CO—, wherein CO represents a carbonyl group and R₃ is a saturated or an unsaturated (mono- or polyunsaturated) hydrocarbon chain having from 1 to 25 carbons; and combinations thereof.

6. The composition according to claim 5, wherein said amino terminal capping group R₁ is lipoic acid (Lip).

7. The composition according to claim 5, wherein said amino terminal capping group R₁ is the acyl group having the formula R₃—CO—, wherein CO represents a carbonyl group and R₃ is a saturated or an unsaturated (mono- or polyunsaturated) hydrocarbon chain having from 1 to 25 carbons.

8. The composition according to claim 7, wherein said R₃ is a saturated or unsaturated hydrocarbon chain having 1 to 22 carbons.

9. The composition according to claim 7, wherein said acyl group is the acyl form of an acid selected from the group consisting of: acetic acid, caprylic acid (C8:0), capric acid (C10:0), lauric acid (C12:0), myristic acid (C14:0), palmitic acid (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22:1), docosatetraenoic acid (C22:4-6), docosapentaenoic acid (C22:5-6), docosapentaenoic acid (C22:5-3), docosahexaenoic acid (C22:6-3), and nervonic acid (C24:1-9).

10. The composition according to claim 9, wherein said acyl group is the acyl from of an acid selected from the group consisting of acetic acid, palmitic acid, and docosahexaenoic acid (DHA).

11. The composition according to any of claims 1-4, wherein said R₂, when present, is a primary amine or a secondary amine.

12. The composition according to any of claims 1-4, wherein said single amino acid based compound is a salt.

13. A method of upregulating the level of expression of a superoxide dismutase gene, a catalase gene, or both, in cells or tissues of a mammal comprising contacting the cells or tissues of a mammal with a composition according to any one of claims 1-4 in an amount effective to upregulate expression of an antioxidative enzyme.

14. A method of counteracting the oxidative effects of reactive oxygen species and free radicals in cells or tissues of a mammal comprising contacting the cells or tissues of a mammal with a composition comprising a single amino acid based compound having the formula:
R₁-Xaa-R₂ (SEQ ID NO:1),
wherein:
R₁ is absent or is an amino terminal capping group;
Xaa is any amino acid, or derivative thereof, that upregulates expression of a gene encoding an antioxidative enzyme;
R₂ is absent or is a carboxy terminal capping group; and
wherein said single amino acid compound upregulates expression of a gene encoding an antioxidative enzyme.

15. The method according to claim 14, wherein said antioxidative enzyme is superoxide dismutase (SOD) or catalase (CAT).

16. The method according to claim 14, wherein said Xaa is selected from the group consisting of L-aspartic acid, D-aspartic acid, L-asparagine, D-asparagine, L-glutamic acid, D-glutamic acid, L-glutamine, D-glutamine, and derivatives thereof.

17. The method according to claim 16, wherein Xaa is L-aspartic acid, L-asparagine, or derivatives thereof.

18. The method according to claim 14, wherein R₁, when present, is selected from the group consisting of: lipoic acid moiety (Lip); a glucose-3-O-glycolic acid (Gga) moiety; 1 to 6 lysines (SEQ ID NO:2); 1 to 6 arginine residues (SEQ ID NO:2); a lysine and arginine containing peptide of 2-6 amino acid residues (SEQ ID NO:2); an acyl group having the formula R₃—CO—, wherein CO represents a carbonyl group and R₃ is a saturated or an unsaturated (mono- or polyunsaturated) hydrocarbon chain having from 1 to 25 carbons; and combinations thereof.

19. The method according to claim 18, wherein said amino terminal capping group R₁ is the acyl group having the formula R₃—CO—, wherein CO represents a carbonyl group and R₃ is a saturated or an unsaturated (mono- or polyunsaturated) hydrocarbon chain having from 1 to 25 carbons.

20. The method according to claim 19, wherein said R₃ is a saturated or unsaturated hydrocarbon chain having 1 to 22 carbons.

21. The composition according to claim 19, wherein said acyl group is the acyl form of an acid selected from the group consisting of: acetic acid, caprylic acid (C8:0), capric acid (C10:0), lauric acid (C12:0), myristic acid (C14:0), palmitic acid (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22:1), docosatetraenoic acid (C22:4-6), docosapentaenoic acid (C22:5-6), docosapentaenoic acid (C22:5-3), docosahexaenoic acid (C22:6-3), and nervonic acid (C24:1-9).

22. The method according to claim 21, wherein said acyl group is the acyl form of an acid selected from the group consisting of acetic acid, palmitic acid, and docosahexaenoic acid (DHA).

23. The method according to any of claims 14-17, wherein said R₂, when present, is selected from the group consisting of: a primary amine and a secondary amine.

24. The method according to any of claims 14-17, wherein said single amino acid based compound is a salt.

25. A method of upregulating the level of expression of a superoxide dismutase gene, a catalase gene, or both, in cells or tissues of a mammal comprising contacting cells or tissues with a composition according to any one of claims 1, 3, and 4 in an amount effective to upregulate expression of an antioxidative enzyme.

26. A method of reducing or preventing an undesirable elevation in the levels of reactive oxygen species and other free radicals in cells or tissues of a mammal comprising contacting the cells or tissues with a composition according to any one of claims 1, 3, and 4 in an amount effective to upregulate expression of an antioxidative enzyme.

27. A method of treating a disease or condition in a mammal exhibiting an undesirable elevation in levels of reactive oxygen species or other free radicals in cells or tissues of said mammal comprising administering to said mammal a composition comprising a single amino acid based compound having the formula:
R₁-Xaa-R₂ (SEQ ID NO:1),
wherein:
R₁ is absent or is an amino terminal capping group;
Xaa is any amino acid, or derivative thereof, that upregulates expression of a gene encoding an antioxidative enzyme;
R₂ is absent or is a carboxy terminal capping group; and
wherein said single amino acid based compound upregulates expression of a gene encoding an antioxidative enzyme.

28. The method according to claim 27, wherein said Xaa is selected from the group consisting of L-aspartic acid, D-aspartic acid, L-asparagine, D-asparagine, L-glutamic acid, D-glutamic acid, L-glutamine, D-glutamine, and derivatives thereof.

29. The method according to claim 27, wherein said antioxidative enzyme is superoxide dismutase (SOD) or catalase (CAT).

30. The method according to claim 27, wherein said disease or condition is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

31. The method according to claim 30, wherein said disease or condition is neurodegenerative disease.

32. The method according to claim 31, wherein said neurodegenerative disease is is selected from the group consisting of Huntington's disease, Parkinson's disease, and amyotrophic lateral sclerosis.

33. The method according to claim 27, wherein said disease or condition is a disease or condition related to the aging process.

34. The method according to claim 33, wherein said disease or condition related to the aging process is selected from the group consisting of decreased cognitive function, decreased motor function, senility, Alzheimer's disease, premature aging, and decreased life expectancy.

35. The method according to claim 27, wherein R₁, when present, is selected from the group consisting of: lipoic acid moiety (Lip); a glucose-3-O-glycolic acid (Gga) moiety; 1 to 6 lysines (SEQ ID NO:2); 1 to 6 arginine residues (SEQ ID NO:2); a lysine and arginine containing peptide of 2-6 amino acid residues (SEQ ID NO:2); an acyl group having the formula R₃—CO—, wherein CO represents a carbonyl group and R₃ is a saturated or an unsaturated (mono- or polyunsaturated) hydrocarbon chain having from 1 to 25 carbons; and combinations thereof.

36. The method according to claim 35, wherein said amino terminal capping group R₁ is the acyl group having the formula R₃—CO—, wherein CO represents a carbonyl group and R₃ is a saturated or an unsaturated (mono- or polyunsaturated) hydrocarbon chain having from 1 to 25 carbons.

37. The method according to claim 36, wherein said R₃ is a saturated or unsaturated hydrocarbon chain having 1 to 22 carbons.

38. The method according to claim 36, wherein said acyl group is the acyl form of an acid selected from the group consisting of: acetic acid, caprylic acid (C8:0), capric acid (C10:0), lauric acid (C12:0), myristic acid (C14:0), palmitic acid (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22:1), docosatetraenoic acid (C22:4-6), docosapentaenoic acid (C22:5-6), docosapentaenoic acid (C22:5-3), docosahexaenoic acid (C22:6-3), and nervonic acid (C24:1-9).

39. The method according to claim 38, wherein said acyl group is the acyl form of an acid selected from the group consisting of acetic acid, palmitic acid, and docosahexaenoic acid (DHA).

40. The method according to claim 27, wherein said R₂, when present, is a primary amine or a secondary amine.

41. The method according to claim 27, wherein said single amino acid based compound is a salt.

42. A pharmaceutical composition comprising a single amino acid based compound having the formula:
R₁-Xaa-R₂ (SEQ ID NO:1),
wherein:
R₁ is absent or is an amino terminal capping group;
Xaa is any amino acid, or derivative thereof, that upregulates expression of a gene encoding an antioxidative enzyme; and
R₂ is absent or is a carboxy terminal capping group.

43. The pharmaceutical composition according to claim 42, wherein said antioxidative enzyme is superoxide dismutase (SOD) or catalase (CAT).

44. The pharmaceutical composition according to claim 42, wherein said Xaa is selected from the group consisting of L-aspartic acid, D-aspartic acid, L-asparagine, D-asparagine, L-glutamic acid, D-glutamic acid, L-glutamine, D-glutamine, and derivatives thereof.

45. The pharmaceutical composition according to any of claims 42, wherein R₁, when present, is selected from the group consisting of: lipoic acid moiety (Lip); a glucose-3-O-glycolic acid (Gga) moiety; 1 to 6 lysines (SEQ ID NO:2); 1 to 6 arginine residues (SEQ ID NO:2); a lysine and arginine containing peptide of 2-6 amino acid residues (SEQ ID NO:2); an acyl group having the formula R₃—CO—, wherein CO represents a carbonyl group and R₃ is a saturated or an unsaturated (mono- or polyunsaturated) hydrocarbon chain having from 1 to 25 carbons; and combinations thereof.

46. The pharmaceutical composition according to claim 42, wherein said amino terminal capping group R₁ is lipoic acid (Lip).

47. The pharmaceutical composition according to claim 42, wherein said amino terminal capping group R₁ is the acyl group having the formula R₃—CO—, wherein CO represents a carbonyl group and R₃ is a saturated or an unsaturated (mono- or polyunsaturated) hydrocarbon chain having from 1 to 25 carbons.

48. The pharmaceutical composition according to claim 47, wherein said acyl group is the acyl form of an acid selected from the group consisting of: acetic acid, caprylic acid (C8:0), capric acid (C10:0), lauric acid (C12:0), myristic acid (C14:0), palmitic acid (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22:1), docosatetraenoic acid (C22:4-6), docosapentaenoic acid (C22:5-6), docosapentaenoic acid (C22:5-3), docosahexaenoic acid (C22:6-3), and nervonic acid (C24:1-9).

49. The pharmaceutical composition according to claim 47, wherein said acyl group is the acyl from of an acid selected from the group consisting of acetic acid, palmitic acid, and docosahexaenoic acid (DHA).

50. The pharmaceutical composition according to any of claim 42, wherein said R₂, when present, is a primary amine or a secondary amine.

51. The pharmaceutical composition according to claim 42, wherein said single amino acid based compound is a salt.

52. A method of upregulating the level of expression of at least one antioxidative enzyme in cells or tissues of a mammal comprising contacting the cells or tissues of a mammal with the pharmaceutical composition according to claim 42 in an amount effective to upregulate expression of an antioxidative enzyme.

53. The method according to claim 52, wherein the antioxidative enzyme is selected from the group consisting of superoxide dismutase and catalase.

54. The method of treating or preventing a disease or condition in a mammal, comprising administering to such mammal an effective amount of the pharmaceutical composition according to claim 42.

55. The method according to claim 54, wherein said disease or condition is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

56. The method according to claim 54, wherein said disease or condition is neurodegenerative disease.

57. The method according to claim 56, wherein said neurodegenerative disease is is selected from the group consisting of Huntington's disease, Parkinson's disease, and amyotrophic lateral sclerosis.

58. The method according to claim 54, wherein said disease or condition is a disease or condition related to the aging process.

59. An isolated pharmaceutical composition comprising a single amino acid based compound having the formula:
R₁-Xaa-R₂ (SEQ ID NO:1),
wherein:
R₁ is absent or is an amino terminal capping group;
Xaa is any amino acid, or derivative thereof, that upregulates expression of a gene encoding an antioxidative enzyme; and
R₂ is absent or is a carboxy terminal capping group.

60. The pharmaceutical composition according to claim 59, wherein said antioxidative enzyme is superoxide dismutase (SOD) or catalase (CAT).

61. The pharmaceutical composition according to claim 59, wherein said Xaa is selected from the group consisting of L-aspartic acid, D-aspartic acid, L-asparagine, D-asparagine, L-glutamic acid, D-glutamic acid, L-glutamine, D-glutamine, and derivatives thereof.

62. The pharmaceutical composition according to any of claims 59, wherein R₁, when present, is selected from the group consisting of: lipoic acid moiety (Lip); a glucose-3-O-glycolic acid (Gga) moiety; 1 to 6 lysines (SEQ ID NO:2); 1 to 6 arginine residues (SEQ ID NO:2); a lysine and arginine containing peptide of 2-6 amino acid residues (SEQ ID NO:2); an acyl group having the formula R₃—CO—, wherein CO represents a carbonyl group and R₃ is a saturated or an unsaturated (mono- or polyunsaturated) hydrocarbon chain having from 1 to 25 carbons; and combinations thereof.

63. The pharmaceutical composition according to claim 59, wherein said amino terminal capping group R₁ is lipoic acid (Lip).

64. The pharmaceutical composition according to claim 59, wherein said amino terminal capping group R₁ is the acyl group having the formula R₃—CO—, wherein CO represents a carbonyl group and R₃ is a saturated or an unsaturated (mono- or polyunsaturated) hydrocarbon chain having from 1 to 25 carbons.

65. The pharmaceutical composition according to claim 59, wherein said acyl group is the acyl form of an acid selected from the group consisting of: acetic acid, caprylic acid (C8:0), capric acid (C10:0), lauric acid (C12:0), myristic acid (C14:0), palmitic acid (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22:1), docosatetraenoic acid (C22:4-6), docosapentaenoic acid (C22:5-6), docosapentaenoic acid (C22:5-3), docosahexaenoic acid (C22:6-3), and nervonic acid (C24:1-9).

66. The pharmaceutical composition according to claim 64, wherein said acyl group is the acyl from of an acid selected from the group consisting of acetic acid, palmitic acid, and docosahexaenoic acid (DHA).

67. The pharmaceutical composition according to any of claim 59, wherein said R₂, when present, is a primary amine or a secondary amine.

68. The pharmaceutical composition according to claim 59, wherein said single amino acid based compound is a salt.

69. A method of upregulating the level of expression of at least one antioxidative enzyme in cells or tissues of a mammal comprising contacting the cells or tissues of a mammal with the pharmaceutical composition according to claim 59 in an amount effective to upregulate expression of an antioxidative enzyme.

70. The method according to claim 69, wherein the antioxidative enzyme is selected from the group consisting of superoxide dismutase and catalase.

71. The method of treating or preventing a disease or condition in a mammal, comprising administering to such mammal an effective amount of the pharmaceutical composition according to claim 59.

72. The method according to claim 71, wherein said disease or condition is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

73. The method according to claim 71, wherein said disease or condition is neurodegenerative disease.

74. The method according to claim 73, wherein said neurodegenerative disease is is selected from the group consisting of Huntington's disease, Parkinson's disease, and amyotrophic lateral sclerosis.

75. The method according to claim 71, wherein said disease or condition is a disease or condition related to the aging process.

76. A pharmaceutical composition consisting essentially of at least one single amino acid based compound having the formula:
R₁-Xaa-R₂ (SEQ ID NO:1),
wherein:
R₁ is absent or is an amino terminal capping group;
Xaa is any amino acid, or derivative thereof, that upregulates expression of a gene encoding an antioxidative enzyme; and
R₂ is absent or is a carboxy terminal capping group.

77. The pharmaceutical composition according to claim 76, wherein said antioxidative enzyme is superoxide dismutase (SOD) or catalase (CAT).

78. The pharmaceutical composition according to claim 76, wherein said Xaa is selected from the group consisting of L-aspartic acid, D-aspartic acid, L-asparagine, D-asparagine, L-glutamic acid, D-glutamic acid, L-glutamine, D-glutamine, and derivatives thereof.

79. The pharmaceutical composition according to any of claims 76, wherein R₁, when present, is selected from the group consisting of: lipoic acid moiety (Lip); a glucose-3-O-glycolic acid (Gga) moiety; 1 to 6 lysines (SEQ ID NO:2); 1 to 6 arginine residues (SEQ ID NO:2); a lysine and arginine containing peptide of 2-6 amino acid residues (SEQ ID NO:2); an acyl group having the formula R₃—CO—, wherein CO represents a carbonyl group and R₃ is a saturated or an unsaturated (mono- or polyunsaturated) hydrocarbon chain having from 1 to 25 carbons; and combinations thereof.

80. The pharmaceutical composition according to claim 76, wherein said amino terminal capping group R₁ is lipoic acid (Lip).

81. The pharmaceutical composition according to claim 76, wherein said amino terminal capping group R₁ is the acyl group having the formula R₃—CO—, wherein CO represents a carbonyl group and R₃ is a saturated or an unsaturated (mono- or polyunsaturated) hydrocarbon chain having from 1 to 25 carbons.

82. The pharmaceutical composition according to claim 79, wherein said acyl group is the acyl form of an acid selected from the group consisting of: acetic acid, caprylic acid (C8:0), capric acid (C10:0), lauric acid (C12:0), myristic acid (C14:0), palmitic acid (C16:0), palmitoleic acid (C16:1), C16:2, stearic acid (C18:0), oleic acid (C18:1), vaccenic acid (C18:1-7), linoleic acid (C18:2-6), α-linolenic acid (C18:3-3), eleostearic acid (C18:3-5), β-linolenic acid (C18:3-6), C18:4-3, gondoic acid (C20:1), C20:2-6, dihomo-γ-linolenic acid (C20:3-6), C20:4-3, arachidonic acid (C20:4-6), eicosapentaenoic acid (C20:5-3), docosenoic acid (C22:1), docosatetraenoic acid (C22:4-6), docosapentaenoic acid (C22:5-6), docosapentaenoic acid (C22:5-3), docosahexaenoic acid (C22:6-3), and nervonic acid (C24:1-9).

83. The pharmaceutical composition according to claim 79, wherein said acyl group is the acyl from of an acid selected from the group consisting of acetic acid, palmitic acid, and docosahexaenoic acid (DHA).

84. The pharmaceutical composition according to any of claim 76, wherein said R₂, when present, is a primary amine or a secondary amine.

85. The pharmaceutical composition according to claim 76, wherein said single amino acid based compound is a salt.

86. A method of upregulating the level of expression of at least one antioxidative enzyme in cells or tissues of a mammal comprising contacting the cells or tissues of a mammal with the pharmaceutical composition according to claim 76 in an amount effective to upregulate expression of an antioxidative enzyme.

87. The method according to claim 86, wherein the antioxidative enzyme is selected from the group consisting of superoxide dismutase and catalase.

88. The method of treating or preventing a disease or condition in a mammal, comprising administering to such mammal an effective amount of the pharmaceutical composition according to claim 86.

89. The method according to claim 88, wherein said disease or condition is selected from the group consisting of cerebral ischemia, myocardial infarct, renal reperfusion, atherosclerosis, head trauma, brain trauma, spinal cord trauma, oxygen toxicity in premature infants, neurodegenerative disease, arthritis, inflammation, diabetes, ulcerative colitis, cancer, Down syndrome, macular degeneration, cataracts, schizophrenia, epilepsy, septic shock, polytraumatous shock, burn injuries, radiation-induced elevation of reactive oxygen species or other free radicals, and drug-induced elevation of reactive oxygen species or other free radicals.

90. The method according to claim 88, wherein said disease or condition is neurodegenerative disease.

91. The method according to claim 90, wherein said neurodegenerative disease is is selected from the group consisting of Huntington's disease, Parkinson's disease, and amyotrophic lateral sclerosis.

92. The method according to claim 88, wherein said disease or condition is a disease or condition related to the aging process.

93. A method of preparing a pharmaceutical composition useful for upregulating expression of a gene encoding an antioxidative enzyme, comprising mixing at least one single amino acid based compound having the formula:
R1-Xaa-R2 (SEQ ID NO:1),
with a suitable vehicle, wherein:
R1 is absent or is an amino terminal capping group;
Xaa is any amino acid, or derivative thereof, that upregulates expression of a gene encoding an antioxidative enzyme; and
R2 is absent or is a carboxy terminal capping group.

94. The method as described in claim 93, wherein the suitable vehicle is a pharmaceutically acceptable excipient.

95. The method as described in claim 93, wherein the vehicle is a salt.

96. The method as described in claim 93, wherein the vehicle is obtained from a natural source.
